(19) United States
(12) Patent Application Publication
(10) Pub. No.: US 2015/0328377 A1
(43) Pub. Date: Nov. 19, 2015

(54) COMPOSITION OF D-ALPHA HYDROXY ACIDS AND ANTIMICROBIALS

(71) Applicant: BACTERIN INTERNATIONAL, INC., Belgrade, MT (US)

(72) Inventor: Mark SCHALLENBERGER, Bozeman, MT (US)

(21) Appl. No.: 14/758,099

(22) PCT Filed: Dec. 31, 2013
(86) PCT No.: PCT/US13/78539

§ 371 Date: Jun. 26, 2015

Related U.S. Application Data
(60) Provisional application No. 61/747,743, filed on Dec. 31, 2012.

(51) Int. Cl. A61L 31/16; A01N 47/44; A01N 43/90; A61L 31/10; A61L 31/08; A61L 31/14; A61L 27/28; A61L 27/54; A01N 37/36; A01N 37/18

(52) U.S. Cl. CPC A61L 31/16; A01N 37/36; A01N 47/44; A01N 43/90; A01N 37/18; A61L 31/08; A61L 31/148; A61L 27/28; A61L 27/54; A61L 31/10; A61L 2300/404; A61L 2300/45

(57) ABSTRACT

The present invention relates to an antimicrobial composition, which is a synergistic combination comprising an anti-microbial agent and a polymer or monomer comprising D-alpha hydroxy acid or a polymer capable of releasing a D-alpha hydroxy acid monomer. The present invention also relates to methods for using and applying the same.

### CROSS REFERENCE TO RELATED APPLICATIONS

This application claims priority under 35 U.S.C. §119(e) to U.S. Provisional Patent Application Ser. No. 61/747,743 filed on Dec. 31, 2012, which is incorporated herein in its entirety by reference.

### FIELD OF THE INVENTION

This invention relates to an antimicrobial composition comprised of a D-alpha hydroxy acid and an antimicrobial agent, the combination of which possess synergistic antimicrobial activity.

### BACKGROUND

In order to minimize the risk of and treat bacterial and fungal-related illness, a variety of antimicrobial/bioactive agents have been employed. Although selected agents have proven abilities to limit disease and inhibit microbial growth, there remains a need for improved infection treatment and control.

Antimicrobial synergy, in which two or more agents are more efficacious together than the additive effect of the two agents, has proven to be effective in controlling microbial diseases and is routinely used in clinical practice. The term “more efficacious” may be described as a combination of agents with more potent activity, activity at lower concentrations, increased sustained activity, and/or inhibiting the emergence of resistance compared to either agent alone. Previously described effective synergistic combinations include rifampin and minocycline; rifampin and clindamycin; rifampin and novobiocin; silver and chlorhexidine; and trimethoprim and sulphmethoxazole.

In addition to general bacterial and fungal-related illnesses, implant-associated infection remains a significant clinical challenge for patients with an implanted medical device. These infections are particularly difficult to treat and often require removal of the infected implant. In order to minimize the risk of implant-associated infections, a variety of antimicrobial/bioactive agents have been employed on the surface of medical devices. Although selected agents have proven abilities to limit disease and inhibit microbial growth, there remains a need for improved infection control.

### SUMMARY

While several synergistic antimicrobial compositions exist for general antimicrobial therapy and medical device surface treatment, it remains particularly advantageous and commercially desirable to obtain new compositions with improved antimicrobial activity.

An aspect of the present invention includes article comprising a physical object and an antimicrobial composition on one or more surfaces of the physical object, wherein the antimicrobial composition comprises a synergistic combination of a D-alpha hydroxy acid and one or more antimicrobial agents.

An aspect of the present invention includes a method of inhibiting microbial growth on one or more surfaces of a physical object by applying to the surface an antimicrobial composition comprising a synergistic combination of a D-alpha hydroxy acid and one or more antimicrobial agents.

An aspect of the present invention includes a method to treat or prevent disease in an animal comprising administering an antimicrobial composition comprising a synergistic combination of a D-alpha hydroxy acid and an antimicrobial agent to the animal.

An aspect of the present invention includes a method of inhibiting microbial growth on one or more surfaces comprising applying an antimicrobial combination comprising a synergistic combination of a D-alpha hydroxy acid and an antimicrobial agent to the one or more surfaces.

An aspect of the present invention includes an antimicrobial composition comprising a synergistic combination of a D-hydroxy acid and one or more antimicrobial agents.

These and other embodiments are discussed in further detail and are readily apparent in view of the following description.

### BRIEF DESCRIPTION OF THE FIGURES

FIG. 1 illustrates the total chlorhexidine elution from coated pins.

FIG. 2 illustrates the log transformation of chlorhexidine within polymeric film over time.

FIG. 3 illustrates the total rifampin and minocycline elution from coated pins over time.

FIG. 4 illustrates the log transformation of rifampin within polymeric film plotted over time.

FIG. 5 illustrates the log transformation of minocycline within polymeric film plotted over time.

FIG. 6 illustrates the ZOIs for coated wires over time.

FIG. 7 illustrates the in vivo efficacy of coated wires compared to uncoated wires.

### DETAILED DESCRIPTION OF THE INVENTION

The present invention is directed to antimicrobial compositions that include a D-alpha hydroxy acid and one or more antimicrobial agents, the combination of which possesses synergistic antimicrobial activity.

An advantage of the present invention is the improved antimicrobial activity of the composition of the D-alpha hydroxy acid and the antimicrobial agent, or a combination of antimicrobial agents, compared to the additive effect of the D-alpha hydroxy acid and the antimicrobial agent, or combination of antimicrobial agents. This improved activity may be observed through increased bactericidal potency, equal activity at lower concentrations of the antimicrobial agent, having longer sustained activity, and/or by inhibiting the emergence of resistance to the antimicrobial agent.

“Antimicrobial agent” as used herein, broadly includes, but is not limited to, antibiotics, antimicrobials, antiseptics, and antifungals and combinations thereof. The terms antimicrobial agents and bioactive substances are used interchangeably herein. Examples include, but are not limited to, bisbiguanides (including chlorhexidine and alexidine), silver nanoparticles, silver nitrate, silver oxide, silver salts, silver sulfadiazine, silver zeolites, triclosan, antifolates, aminoglycosides, carbapenems, cephalosporins, fluoroquinolines, glycopeptides, macrolides, monobactams, oxazolidones, penicillins, rifamycins, sulfonamides and tetracyclines and combinations thereof. Antimicrobial agents may be used individually or as a mixture of multiple antimicrobial agents. The antimicrobial agent may be in the form of a salt. Furthermore, antimicrobial agents, for example bisbiguanides, may be used in their monomer form, or polymer form.

In some embodiments, the antimicrobial agent used in this invention may be a combination of rifamycins and tetracyclines. In some embodiments, the rifamycins may be rifampin and/or a salt thereof and the tetracycline may be minocycline and/or a salt thereof.

“Biodegradable polymer” as used herein, is broadly defined as any polymer being capable of being broken down by natural biological or environmental processes such as the hydrolysis of bonds between monomers in the case of polyhydroxy acid polymers. Through this degradation process, the polymer in whole or in part releases the monomers that the polymer is comprised of. Additionally, this phenomenon may be used to control the release of various bioactive substances. In this sense, the bioactive substance may be dispersed within the polymer and as the polymer degrades it may expose the bioactive agent to the medium in which the polymer may be exposed allowing the bioactive agent to be released into the medium. Suitable biodegradable polymers can be formed by polymerization in whole or in part of D-alpha hydroxy acid derived monomers, which include but are not limited to polylactic acids or polylactides or interpolymers and copolymers thereof. Biodegradable polymers may be used as a polymeric base material as described below. In some embodiments, the polymer chains are formed from D-alpha hydroxy acid derived monomers, while in other embodiments, the D-alpha hydroxy acid monomers are dispersed among a polymer chain, where the polymer is not necessarily a polymer of the D-alpha hydroxy acid monomers. Suitable polymers include, but are not limited to, polycaprolactones, polyethylene glycols, polyhydroxyalkanoates, polyesteramides, polyglycolides, polyorthoesters, polyoxazolines, polyurethanes and combinations thereof.

In some embodiments of the present invention, the polymeric base material may be comprised of a polymer formed by the polymerization in whole or in part of the D-alpha hydroxy acid derived monomers selected from the group of polylactic acid, polylactides, interpolymers and copolymers thereof. In some embodiments, the antimicrobial agent may be bisbiguanides.

The process of controlling the release of an antimicrobial agent or bioactive substance based primarily on polymer degradation is in contrast to a system in which the release of the bioactive substance is controlled primarily by diffusion of the bioactive agent from the polymer. Importantly, depending on the physicochemical properties of a bioactive agent and a given biodegradable polymer, controlled release may occur through either polymer degradation or bioactive diffusion. A mechanism of controlled release based on degradation may be determined by plotting the log of the total quantity of bioactive agent left within the polymer against time. If the resulting plot is linear, it is demonstrated that the mechanism of controlled release of the bioactive agent occurs through degradation of the polymer. Thus, the mechanism of controlled release (degradation or diffusion) for a combination of a given polymer and antimicrobial agent(s) may be determined without undue experimentation.

It is well known in the art that the rate of biodegradation of a polymeric system can be controlled by modifying the chemical makeup of the polymeric system. For example, polymeric systems with shorter polymer chain lengths typically biodegrade faster than similar systems with longer chain lengths. Similarly, more hydrophilic polymers typically biodegrade faster than more hydrophobic polymers. Therefore, the rate of polymer biodegradation, and as such the rate of bioactive controlled release, can be easily controlled to match the exact requirements for a given system.

“D-alpha hydroxy acid” as used herein, is broadly defined as the class of organic molecules containing a hydroxy moiety in the alpha position to a carboxylic acid moiety with a D-stereochemistry (symbolized by the D-). L-alpha hydroxy acids possess the same constitutional connectivity with an L stereochemistry (symbolized by the L-) stereochemistry. D-alpha hydroxy acid can be selected from acids, or the ester, salt, amide, or other derivatives of the group consisting of D-lactic acid, D-glycolic acid, D-tartaric acid, D-mandelic acid, D-succinic acid, D-benzylic acid, D-1-hydroxy 1 cyclohexane carboxylic acid, D-2-hydroxy-1-(2-tetrahydrofuranyl) ethanoic acid, D-2-hydroxy-2-(2-furanyl) ethanoic acid, D-2-hydroxy-2-phenylpropionic acid, D-2-hydroxy-2-methylpropionic acid, D-2-hydroxy-2-methylbutanoic acid, D-2-hydroxybutanoic acid, D-2-hydroxypentanoic acid, or mixtures thereof. Preferably, the D-alpha hydroxy acid is D-lactic acid and/or salts thereof. Examples of L-alpha hydroxy acids can be can be selected from acids, or the ester, salt, amide, or other derivatives of the group consisting of L-lactic acid, L-glycolic acid, L-tartaric acid, L-mandelic acid, L-succinic acid, L-benzylic acid, L-1-hydroxy 1 cyclohexane carboxylic acid, L-2-hydroxy-1-(2-tetrahydrofuranyl) ethanoic acid, L-2-hydroxy-2-(2-furanyl) ethanoic acid, L-2-hydroxy-2-phenylpropionic acid, L-2-hydroxy-2-methylpropionic acid, L-2-hydroxy-2-methylbutanoic acid, L-2-hydroxybutanoic acid, L-2-hydroxypentanoic acid, or mixtures thereof. Preferably, the L-alpha hydroxy acid is L-lactic acid and/or salts thereof.

The term “synergistic composition” or “more efficacious” in the context of this antimicrobial composition is the characteristic that the combination of D-alpha hydroxy acid monomer/polymer and the antimicrobial agent(s) has more potent activity, activity at lower concentrations, increased sustained activity, and/or inhibits the emergence of resistance of microorganisms compared to the additive effect of the antimicrobial agent(s) and the D-alpha hydroxy acid monomer/polymer.

An aspect of the present invention comprises applying an antimicrobial composition of the present invention to one or more surfaces of a physical object to inhibit microbial growth or colonization on the object's surface. In some embodiments, the physical object may be one or more surfaces of a medical device, a biological tissue, a table, an industrial surface, a household surface, a medical surface, or other object or surface. Medical devices include, but are not limited to, an instrument, an implant, device, apparatus, tool, combinations thereof or other device, whether reusable, disposable, permanent or temporary. The medical device or biological tissue may be any device used during the diagnosis or treatment of a patient, or the prevention of disease. By way of example, the medical device may be made of a material that is metal, plastic, glass, polymeric, elastomeric, combinations thereof or any other suitable material. By way of example, biological tissue may include, but is not limited to, allograft tissue, autograft tissue, xenograft tissue and combinations thereof. Specific tissue types include, but are not limited to, cortical bone, cancellous bone, demineralized bone, connective tissue, tendon, pericardium, dermis, accellular dermis, cornea, dura matter, fascia, heart valve, ligament, capsular graft, cartilage, collagen, nerves, placental tissue, and combinations thereof.

The antimicrobial composition can be comprised of a polymeric base material that is capable of releasing D-alpha hydroxy acid, which may be in the monomer form, and an antimicrobial agent. A solution comprising the antimicrobial composition and a casting solvent may be used to apply the antimicrobial composition to the physical object. The casting solvent can be selected from the group consisting of, but are not limited to, acetone, acetonitrile, chloroform, diethyl ether, dimethylacetamide, dimethylformamide, dimethylsulfoxide, ethanol, ethyl acetate, hexafluoroisopropanol, hexane, methanol, methylene chloride, tetrahydrofuran, toluene, water and any combinations of two or more of the foregoing.

The antimicrobial composition may be applied to the physical object using any suitable method, including but not limited to dipping, spraying, soaking, brushing, submerging or other suitable method. The viscosity of the antimicrobial composition may be between about 0.5 cP and about 500 cP. If the physical object is submerged, dipped or soaked, or other similar method, the physical object may be removed from the solution containing the antimicrobial composition at a controlled rate. The controlled rate may be between about 0.01 cm/sec to about 50 cm/sec. Following the application, the antimicrobial composition may be cooled at a temperature between about 15° C. to about 50° C., under ambient or reduced pressure (between about 1 nTorr and about 760 Torr) for at least one minute. If a casting solvent was used to apply the antimicrobial composition to the physical object, then it may be evaporated at a temperature between 15° C. to about 50° C. for between about 2 minutes to about 7 days, in some embodiments at ambient conditions, for between about 24 hours to about 48 hours under ambient or reduced pressure (between about 1 nTorr and about 760 Torr).

In some embodiments, the antimicrobial composition may be covalently bonded to one or more of the surfaces of the physical object. In other embodiments, the antimicrobial composition may be ionically bound to one or more of the surfaces of the physical object. In some embodiments, the antimicrobial composition may be passively adsorbed on one or more surfaces of the physical object. In still other embodiments, the antimicrobial composition may be dispersed on one or more surfaces of the physical object.

The polymeric base material used in different aspects and embodiments of this invention may be formed by the polymerization in whole or in part of the D-alpha hydroxy acid derived monomers. In some embodiments, the polymeric base material may be capable of releasing D-alpha hydroxy acid monomers and/or salts or derivatives thereof. In some embodiments, the polymeric base material may be poly(D,L-lactide-co-glycolide) with a molecular weight between about 1 to about 200 kDa, about 50 to about 150 kDa, or about 75 kDa to about 125 kDa. The mole percentage of D,L-lactide in the polymeric base material may be about 1% to about 100%, about 25% to about 90%, about 40% to about 80%, or about 50% to about 75%. The ratio of the D-form of the lactide to the L-form of the lactide in the polymeric base material may be about 1:100 to about 100:0, about 1:3 to about 3:1, about 1:1. In some embodiments, the polymeric base material may be poly(D,L-lactide-co-glycolide) comprising about 75% D,L lactide, and about 25% glycolide, an about1:1 ratio of D-lactide to L-lactide, where the molecular weight of the polymer is about 112 kDa.

The antimicrobial agent used in different aspects and embodiments of this invention may be dispersed within the polymeric base material at concentrations between about 0.01% by weight to about 50% by weight, about 5% by weight to about 35% by weight, or about 15% by weight to about 25% by weight to the weight of the polymeric base material.

An aspect of the invention comprises the application of a dispersion to the surface of the physical object. The dispersion comprises one or more antimicrobial agents within a polymeric base material. The base material is formed by the polymerization in whole or in part of D-alpha hydroxy acid derived monomers. The viscosity of the dispersion may be between about 0.5 cP and about 500 cP.

In embodiments of the invention, the antimicrobial agent and the amount of D-alpha hydroxy acid can be present, independently, in concentrations between about 0.1 μg/cm² of the surface of the physical object and about 10,000 μg/cm², between about 5 μg/cm² of the surface of the physical object and about 1000 μg/cm², between about 10 μg/cm² of the surface of the physical object and about 500 μg/cm², between about 50 μg/cm² of the surface of the physical object and about 400 μg/cm², between about 100 μg/cm² of the surface of the physical object and about 300 μg/cm². It should be noted that the D-alpha hydroxy acid can be expressed as a concentration of the free acid, but may also encompass equivalent amounts of the acid that are present in the form of a polymer.

Another aspect of the invention employs an antimicrobial composition comprising a particular antimicrobial agent, which may be combined with one or more other antimicrobial agents, and biodegradable polymeric base material, that is formed by the polymerization in whole or in part of D-alpha hydroxy acid derived monomers. The antimicrobial agent, or combination of antimicrobial agents, is primarily released through the biodegradation of the polymeric base material and not through diffusion of the antimicrobial agent(s), such that the release of the D-alpha hydroxy acid and the antimicrobial agent(s) occur at similar rates. In some embodiments, the release rate is between about 0.0001 μg/cm²/hour and about 10,000 μg/cm²/hour.

Another embodiment comprises an antimicrobial composition comprising a polymeric base material formed by the polymerization in whole or in part of the D-alpha hydroxy acid derived monomers, which can be selected from the group of polylactic acid, polylactides, interpolymers and copolymers thereof, and the antimicrobial agent is a combination of one antimicrobial selected from the group of rifamycins and the other selected from the group of tetracyclines.

Another aspect of the invention comprises an antimicrobial composition comprising D-alpha hydroxy acid and an antimicrobial agent. This antimicrobial composition could be used broadly to treat microbial illnesses through topical, oral, parenteral, or any other appropriate administration well known in the art. In some embodiments, the monomeric material is D-alpha hydroxy acid in its monomeric form, and not as a component of a polymer. In this embodiment, the ratio of D-alpha hydroxy acid and the antimicrobial agent can be present in ratios from about 1:99 to about 99:1, about 10:90 to about 90:10, about 20:80 to about 80:20, about 30:70 to about 70:30, 40:60 to about 60:40 or about 50:50, based on weight percent of the active compounds.

Another aspect of the present invention is a method of inhibiting microbial growth on one or more surfaces of a physical object by applying to the surface an antimicrobial composition comprising a synergistic combination of a D-alpha hydroxy acid and an antimicrobial agent. In some embodiments, the antimicrobial agent may be primarily released from the polymeric base material through biodegradation of the polymeric base material and not through diffusion of the antimicrobial agent, such that the release of the D-alpha hydroxy acid and the antimicrobial agent occur at similar rates.

An aspect of the present invention is a method to treat or prevent disease in an animal by administering a synergistic combination of a D-alpha hydroxy acid and an antimicrobial agent to the animal. In some embodiments, the antimicrobial composition may be used as a systemic antimicrobial therapy, a systemic prophylaxis, a local antimicrobial therapy, local prophylaxis, a topical antimicrobial therapy, topical prophylaxis, and combinations thereof. As used herein, the term “animal” includes food production animals (e.g. cattle, pigs, lamb, fowl (chickens, turkeys, etc.), fish, and shelfish, companion animals (e.g. dogs, cats, and horses), working animals (e.g. dogs and horses), and humans. Preferably, the animal is a human.

An advantage of the present invention is that the antimicrobial composition may be used for treatment therapies, including for example, systemic antimicrobial therapy, systemic prophylaxis, topical antimicrobial therapy, topical prophylaxis, local antimicrobial therapy and/or local prophylaxis, or combinations thereof.

An aspect of the present invention is a method of inhibiting microbial growth on one or more surfaces by applying to the surfaces an antimicrobial combination comprising a synergistic combination of a D-alpha hydroxy acid and an antimicrobial agent. In some embodiments, the surface(s) may be selected from the group consisting of a surface of a table, a biological tissue, an industrial surface, a household surface, a medical surface, and combinations thereof.

### EXAMPLES

### Example 1

### Synergy Between D-Alpha Hydroxy Acid and an Antimicrobial Agent

The synergy between a D-alpha hydroxy acid and an antimicrobial agent was determined through a standard microplate biofilm formation assay (Antimicrob Agents Chemother. 2009, 53, 4159-4166—which is incorporated in its entirety by reference). Briefly, individual wells of flat bottomed 96 well plates were incubated with approximately 5×10⁵ colony forming units of S. aureus in about 100 μL of tryptic soy broth supplemented with about 0.25% glucose. Before incubation each well received about 0, about 2, about 20, or about 200 μg/mL L- or D-lactic acids and about 0.0 or about 0.5 μg/mL chlorhexidine (CHX). The plates were incubated for approximately 20 hours at about 37° C. and then the supernatant of the wells was removed. Each well was gently washed 3 times with phosphate buffered saline, and the biofilm was fixed by incubating the plates at about 60° C. for about 1 hour. The biofilm was then stained by incubating at about 37° C. in about 100 μL about 0.5% crystal violet for about 20 minutes. The plates were then thoroughly rinsed with tap water to remove any excess crystal violet and dried. The residual crystal violet was then extracted with about 200 μL of about 33% acetic acid. About 150 μL of the about 33% acetic acid extractant was then diluted with about 2000 μL acetonitrile and the quantity of crystal violet was determined by measuring the absorbance of the resulting solution at about 590 nanometers. The concentration of crystal violet is directly proportional to the amount of biofilm formation in each well. Each set of concentrations was measured at least four times. The average crystal violet concentration was divided by the average crystal violet concentration for a non-treated control (about 0.0 μg/mL CHX and about 0.0 μg/mL lactic acid) to obtain a relative biofilm formation score.

Table 1 is the relative biofilm formation score of D-lactic acid and L-lactic acid with varying amounts of chlorhexidine. All values listed in Table 1 are approximate. As shown, the D-lactic acid showed a statistically significant dose dependent decrease in biofilm formation when combined with chlorhexidine but not by itself The L-lactic acid did not show any statistically significant effects on biofilm formation alone or in combination with chlorhexidine at any concentration. It is highly unexpected that the D-lactic acid and chlorhexidine possess this synergy in inhibiting biofilm formation. It is further unexpected that only the D version of lactic acid possesses this synergy with chlorhexidine and not the L version.

TABLE 1
Relative Biofilm Score

| | D-Lactic Acid | | L-Lactic Acid | |
|---|---|---|---|---|
| μg/mL | 0.0 μg/mL | 0.5 μg/mL | 0.0 μg/mL | 0.5 μg/mL |
| Lactic Acid | CHX | CHX | CHX | CHX |
| 0 | 1.00 ± 0.18 | 0.26 ± 0.04 | 1.00 ± 0.18 | 0.26 ± 0.05 |
| 2 | 1.07 ± 0.24 | 0.19 ± 0.03 | 1.01 ± 0.20 | 0.23 ± 0.05 |
| 20 | 1.11 ± 0.17 | 0.12 ± 0.03 | 1.06 ± 0.18 | 0.33 ± 0.08 |
| 200 | 1.10 ± 0.20 | 0.04 ± 0.01 | 1.16 ± 0.18 | 0.27 ± 0.07 |

### Example 2

### Synergy Between D-Alpha Hydroxy Acid and a Second Antimicrobial Agent

In the same manner as Example 1, the synergy between a D-alpha hydroxy acid and the combination of rifampin and minocycline was determined. Briefly, individual wells of flat bottomed 96 well plates were incubated with approximately 5×10⁵ colony forming units of S. aureus in about 100 μL of tryptic soy broth supplemented with about 0.25% glucose. Before incubation each well received about 0 μg/mL or about 200 μg/mL of L- or D-lactic acid and about 0 ng/mL or about 5 ng/mL each of rifampin and minocycline. The plates were incubated for about 20 hours at about 37° C. and then the supernatant of the wells was removed. The quantity of biofilm formation in each well was determined using the protocol detailed in Example 1.

Table 2 is the relative biofilm formation score of D-lactic acid and L-lactic acid with varying amounts of the mixture of rifampin/minocycline. All values in Table 2 are approximate. As illustrated, the D-lactic acid caused a statistically significant decrease in biofilm formation when combined with rifampin and minocycline but not by itself. Once again the L-lactic acid did not show any reduction in biofilm formation by itself or when combined with rifampin and minocycline.

TABLE 2
Relative Biofilm Score

| | D-Lactic Acid | | L-Lactic Acid | |
|---|---|---|---|---|
| μg/mL | 0.0 ng/mL | 5.0 ng/mL | 0.0 ng/mL | 5.0 ng/mL |
| Lactic Acid | Rif/Min | Rif/Min | Rif/Min | Rif/Min |
| 0 | 1.00 ± 0.24 | 0.47 ± 0.07 | 1.00 ± 0.24 | 0.47 ± 0.07 |
| 200 | 0.92 ± 0.24 | 0.27 ± 0.04 | 1.04 ± 0.15 | 0.43 ± 0.03 |

### Example 3

### Method of Applying a Composition of D-Alpha Hydroxy Acid and an Antimicrobial Agent on the Surface of a Medical Device

The following method was used to provide a medical device with the antimicrobial composition of the invention applied to its surface. Under this embodiment, a dispersion of an antimicrobial agent within a biodegradable polymeric base material that is formed by the polymerization in whole or in part of D-alpha hydroxy acid derived monomers, was applied to the surface of a medical device. The release of the antimicrobial agent was controlled by the biodegradation of the polymeric base material such that the release of the D-alpha hydroxy acid and the antimicrobial agent occurred at similar rates.

The antimicrobial composition comprised about 3.5 grams of poly(D,L-lactide-co-glycolide), which included about 75% D,L lactide (in an about 1:1 ratio of the D and L form) and about 25% glycolide (molecular weight about 112 kDa) and about 0.87 grams of chlorhexidine free base, which were added to a stirring solution of acetonitrile (about 14 mL). The resultant mixture was stirred at about 40° C. until all solids dissolved. The solution was then allowed to cool to about 22° C. Stainless steel pins were then coated by submersion into the coating solution followed by withdrawal from the solution at a controlled rate. After removal from the coating solution the casting solvent was allowed to evaporate from the articles under ambient conditions for between about 24 to about 48 hours. The resulting articles had on their surface about 1044 μg/cm² poly(D,L-lactide-co-glycolide) (equivalent to about 392 μg/cm² D-lactic acid) and about 261 μg/cm² chlorhexidine.

### Example 4

### Release Kinetics of Chlorhexidine from Films

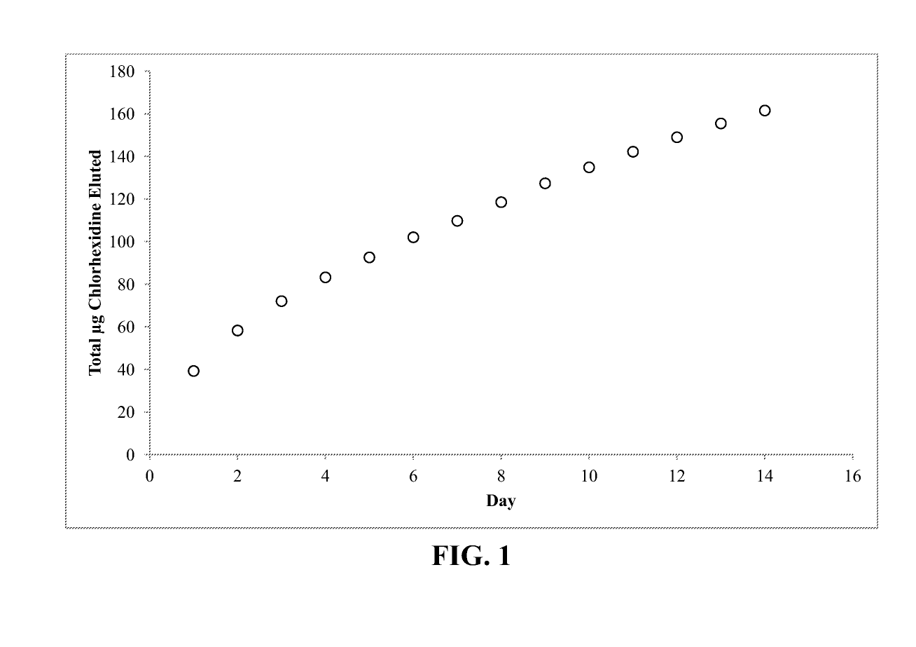

To demonstrate that the release of the antimicrobial agent was controlled by the biodegradation of the polymeric base material such that the release of the D-alpha hydroxy acid and the antimicrobial agent occurred at similar rates, the elution of chlorhexidine from the coated pins generated in Example 3 was measured in phosphate buffered saline for 14 days, changing out the phosphate buffered saline daily. The elution of the chlorhexidine over time is illustrated in FIG. 1.

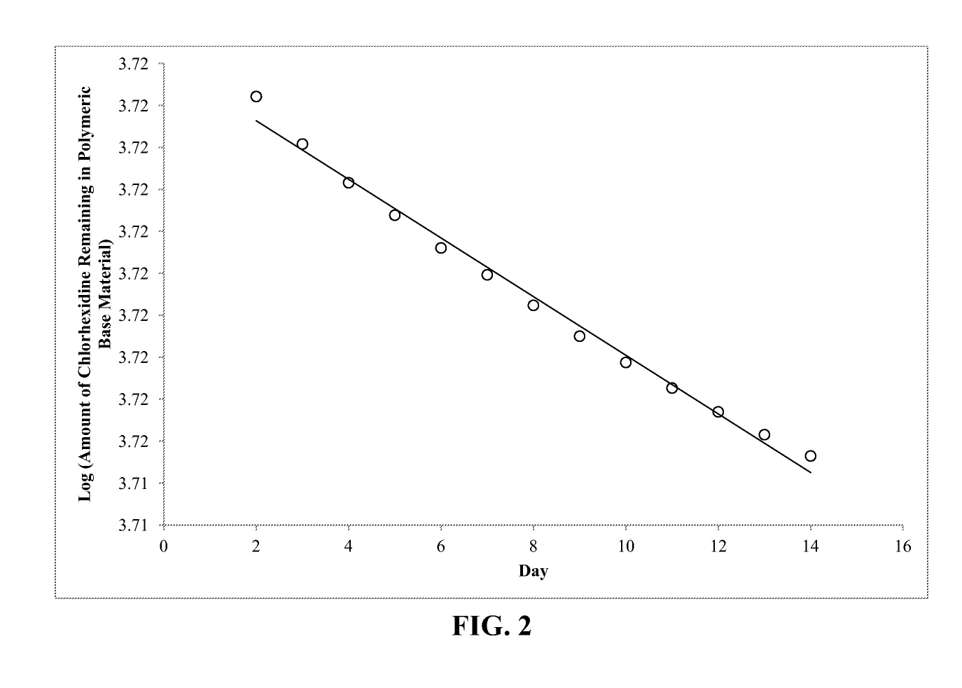

The amount of chlorhexidine remaining in the polymeric base material was calculated, log transformed and plotted over time. The results are illustrated in FIG. 2. As shown, the resulting plot is linear (R² value of about 0.991) indicating a polymer degradation mechanism for release of chlorhexidine from the polymeric base material.

### Example 5

### Second Method of Applying a Composition of D-Alpha Hydroxy Acid and an Antimicrobial Agent on the Surface of a Medical Device

Example 5 illustrates a dispersion of two antimicrobial agents within a biodegradable polymeric base material that is formed by the polymerization in whole or in part of D-alpha hydroxy acid derived monomers which was applied to the surface of a medical device. The release of the antimicrobial agents was controlled by the biodegradation of the polymeric base material such that the release of the D-alpha hydroxy acid and the antimicrobial agents occurred at similar rates.

A mixture comprising about 0.67 grams of poly(D,L-lactide-co-glycolide), which was comprised of about 75% D,L lactide (in an about 1:1 ratio of the D and L form) and 25% glycolide (molecular weight of about 112 kDa), about 0.07 grams of rifampin and about 0.07 grams of minocycline HCl were added to a stirring solution of about 5 mL of acetonitrile: methanol (9:1). The resultant mixture was stirred at about 40° C. until all solids dissolved. The solution was then allowed to cool to about 22° C. Stainless steel pins were coated by submersion into the coating solution followed by withdrawal from the solution at a controlled rate. After the coated pins were removed from the coating solution, the casting solvent was allowed to evaporate from the pins under ambient conditions for between about 24 hours to about 48 hours. The resulting articles had on their surface about 2170 μg/cm² poly(D,L-lactide-co-glycolide) (equivalent to 814 μg/cm² D-lactic acid) and about 217 μg/cm² rifampin and about 217 μg/cm² minocycline.

### Example 6

### Release Kinetics of Rifampin and Minocycline from Films

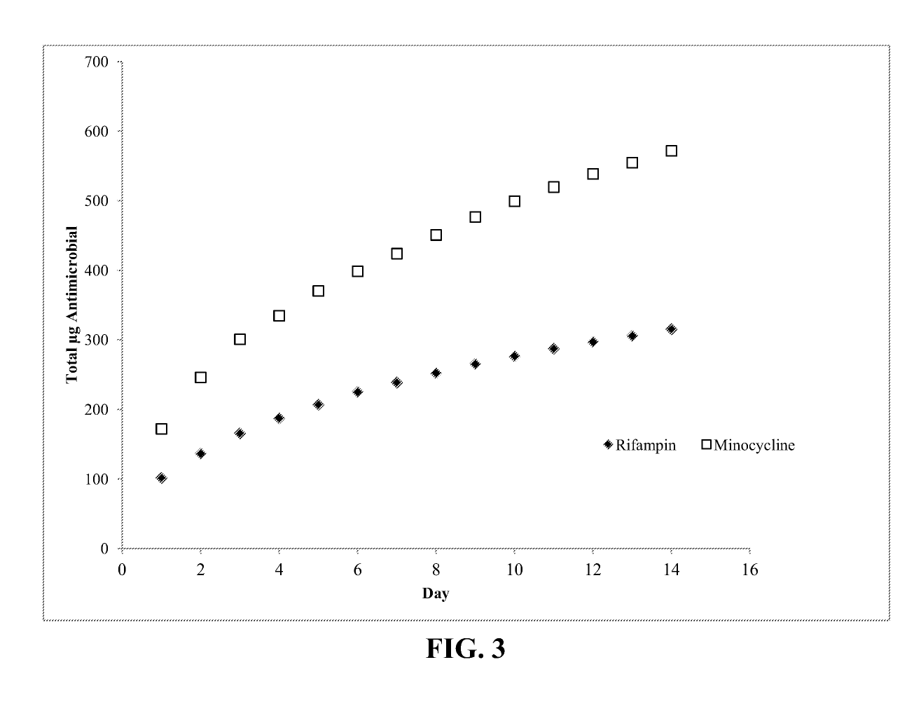

To demonstrate that the release of the antimicrobial agent is controlled by the biodegradation of the polymeric base material such that the release of the D-alpha hydroxy acid and the antimicrobial agent occur at similar rates, the elution rate of rifampin and minocycline from the coated pins generated in Example 5 was measured in phosphate buffered saline for 14 days, changing out the phosphate buffered saline daily. The elution of the rifampin and minocycline is illustrated in FIG. 3.

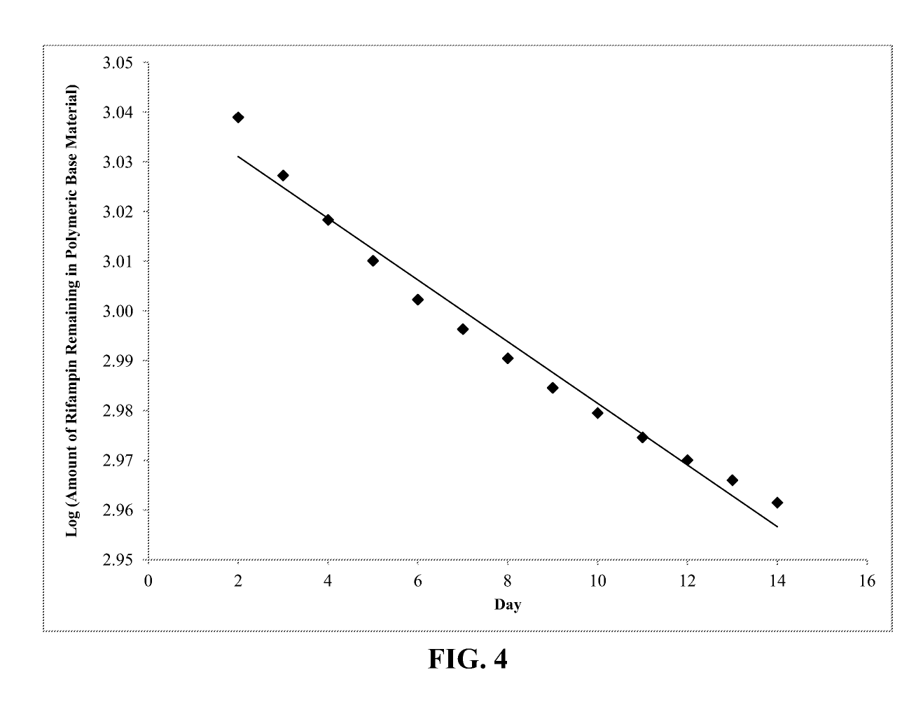

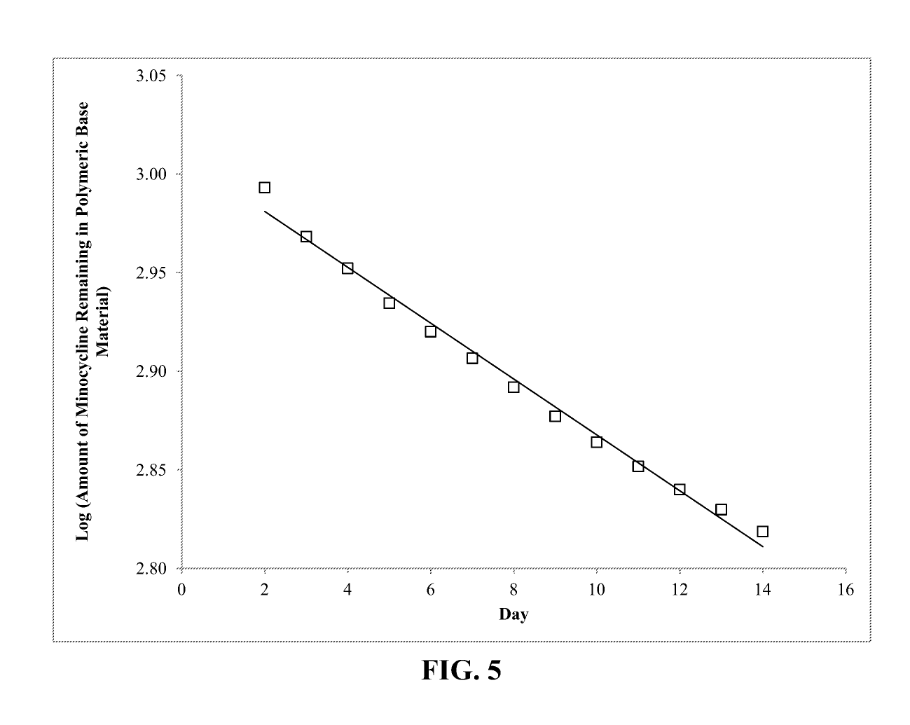

The amount of rifampin and rifampin/minocycline remaining in the polymeric base material was calculated, log transformed and plotted over time. FIG. 4 illustrates the amount of rifampin remaining in the polymeric base over time, while FIG. 5 illustrates the amount of minocycline remaining in the polymeric over time. As shown, the resulting plot is linear (R² value of 0.977 for rifampin and R² value of 0.991 minocycline, respectively) indicating a polymer degradation mechanism of release for rifampin and minocycline from the polymeric base material.

### Example 7

### In vitro Antimicrobial Activity of Coated Pins

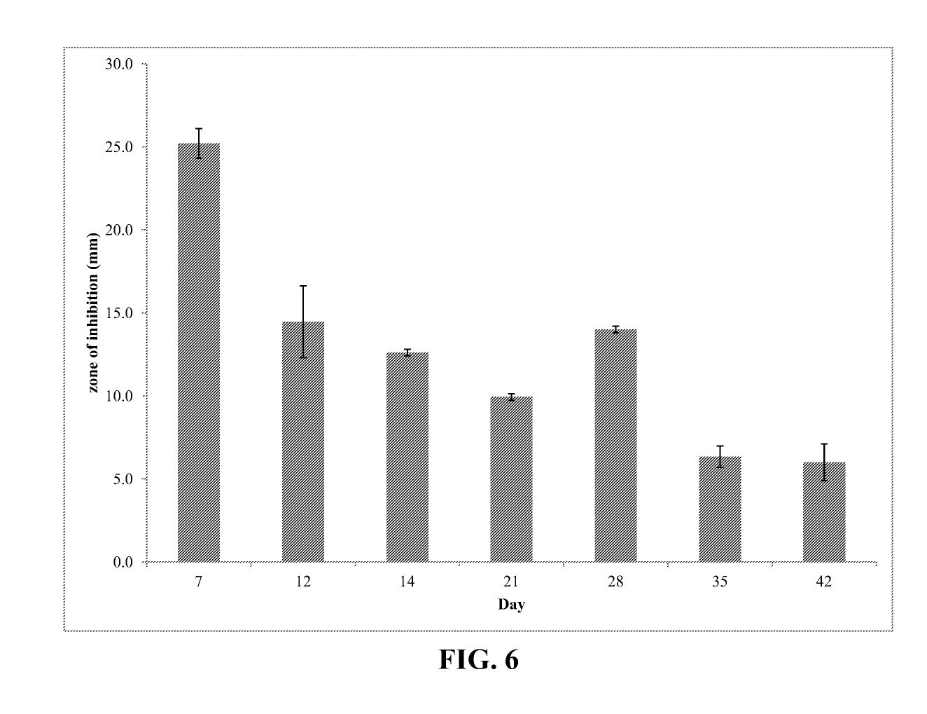

The in vitro antimicrobial activity described in Example 5 was determined by measuring repeat zones of inhibition (ZOIs) against S. aureus. Stainless steel K-wires were coated with the combination of poly(D,L-lactide-co-glycolide), rifampin and minocycline as described in Example 5. The coated wires were placed on a lawn of S. aureus on trypticase soy agar plates and incubated for about 24 hours at about 37° C. Following the incubation, the size of the zone was measured. The coated wires were then placed in phosphate buffered saline at about 37° C. until the next ZOI was performed. ZOIs were determined weekly for 6 weeks and the size of the zone at each week is illustrated in FIG. 6. As illustrated in FIG. 6, the coated wires continued to produce sizeable zones of inhibition for at least 42 days.

### Example 8

### In vivo Antimicrobial Activity of Coated Pins

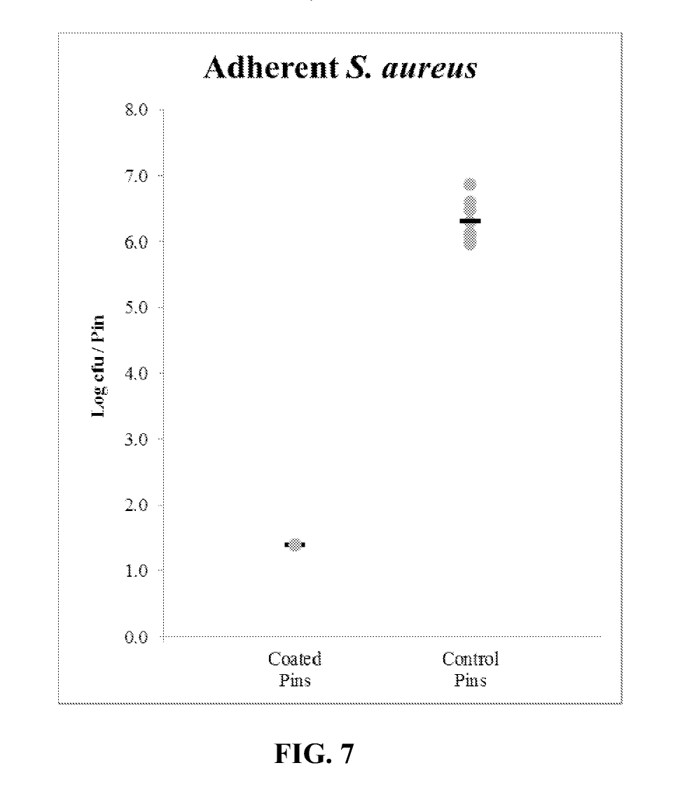

The in vivo antimicrobial activity described in Example 5 was determined through a rabbit model of pin track infection. Stainless steel K-wires were coated with the combination of poly(D,L-lactide-co-glycolide), rifampin and minocycline as described in Example 5. Coated and control (plain stainless steel) K-wires were implanted percutaneously into the tibial metaphysis of New Zealand White rabbits. The surrounding soft tissue was surgically closed and the K-wire skin interface was inoculated with 1×10⁷ colony forming units (cfu) of S. aureus. After seven days, the animals (n=8) were euthanized and the severity of infection was evaluated through the enumeration of adherent bacteria on the surface of the K-wires. During the study, the coating completely inhibited biofilm formation on the surface of the K-wires (limit of detection=3.7×10¹ cfu/cm²), while the non-coated K-wires were colonized with 3.0×10⁶ ±1.5×10⁵ cfu/cm² as illustrated in FIG. 7. This inhibited biofilm formation translates to an about 4.9 log reduction (p<0.0001) in adherent S. aureus. Thus, the synergistic combination was effective at inhibiting biofilm formation on the surface of the K-wires in vivo.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. An article comprising a physical object and an antimicrobial composition on one or more surfaces of the physical object, wherein the antimicrobial composition comprises a synergistic combination of a D-alpha hydroxy acid and one or more antimicrobial agents.

2. The article of claim 1, wherein the physical object is selected from the group consisting of a medical device, a biological tissue, a table, an industrial surface, a household surface, a medical surface, and combinations thereof.

3. The article of claim 2, wherein the physical object is the medical device.

4. The article of claim 3, wherein the medical device is selected from the group consisting of an instrument, an implant, a device, an apparatus, and a tool.

5. The article of claim 3, wherein the medical device is reusable.

6. The article of claim 3, wherein the medical device is disposable.

7. The article of claim 3, wherein the medical device comprises a material selected from the group consisting of metal, plastic, glass, polymeric, elastomeric, and combinations thereof.

8. The article of claim 1, wherein the antimicrobial composition is effective in inhibiting microbial growth on the one or more surfaces of the physical object.

9. The article of claim 1, wherein the synergistic combination of the D-alpha hydroxy acid and the one or more antimicrobial agents is covalently bound to the one or more surfaces of the physical object.

10. The article of claim 1, wherein the synergistic combination of the D-alpha hydroxy acid and the one or more antimicrobial agents is ionically bound to the one or more surfaces of the physical object.

11. The article of claim 1, wherein the synergistic combination of the D-alpha hydroxy acid and the one or more antimicrobial agents is passively adsorbed on the one or more surfaces of the physical object.

12. The article of claim 1, wherein the D-alpha hydroxy acid is within a polymer capable of releasing D-alpha hydroxy acid monomers.

13. The article of claim 12, wherein the polymer is selected from the group consisting of polycaprolactones, polyethylene glycols, polyhydroxyalkanoates, polyesteramides, polyglycolides, polyorthoesters, polyoxazolines, polyurethanes and combinations thereof.

14. The article of claim 12, wherein the one or more antimicrobial agents is dispersed in the polymer.

15. The article of claim 1, wherein the D-alpha hydroxy acid is in the form of a polymeric base material.

16. The article of claim 15, wherein the polymeric base material comprised a polymer of D-alpha hydroxy acid derived monomers.

17. The article of claim 16, wherein the one or more antimicrobial agents is dispersed in the polymer of D-alpha hydroxy acid derived monomers.

18. The article of claim 15, wherein the one or more antimicrobial agents may be primarily released from the polymeric base material by biodegradation of the polymeric base material and not by diffusion.

19. The article of claim 18, wherein the biodegradation of the D-alpha hydroxy acid and the release of the one or more antimicrobial agents occur at similar rates.

20. The article of claim 1, wherein the D-alpha hydroxy acid is in monomeric form.

21. The article of claim 1, wherein the D-alpha hydroxy acid is selected from the group consisting of D-lactic acid, salts thereof and combinations thereof.

22. The article of claim 1, wherein the one or more antimicrobial agents is selected from the group consisting of antibiotics, antimicrobials, antiseptics, antifungals and combinations thereof.

23. The article of claim 1, wherein the one or more antimicrobial agents is selected from the group consisting of bisbiguanide, silver nanoparticles, silver nitrate, silver oxide, silver salts, silver sulfadiazine, silver zeolites, triclosan, antifolates, aminoglycosides, carbapenems, cephalosporins, fluoroquinolines, glycopeptides, macrolides, monobactams, oxazolidones, penicillin, rifamycins, sulfonamide, tetracycline, salts thereof and combinations thereof.

24. The article of claim 23, wherein the one or more antimicrobial agents is a bisbiguanide selected from the group consisting of chlorhexidine, alexidine and combinations thereof.

25. The article of claim 1, wherein the one or more antimicrobial agents is in a form of a salt.

26. The article of claim 1, wherein the one or more antimicrobial agent is a bisbiguanide.

27. The article of claim 23, wherein the one or more antimicrobial agent is a bisbiguanide is in monomeric form.

28. The article of claim 23, wherein the one or more antimicrobial agent is a bisbiguanide is in polymeric form.

29. The article of claim 23, wherein the one or more antimicrobial agent is a bisbiguanide, and wherein the bisbiguanide is chlorhexidine and/or salts thereof.

30. The article of claim 1, wherein the one or more antimicrobial agents comprising an agent selected from the group consisting of rifamycins and an agent selected from the group consisting of tetracyclines.

31. The article of claim 30, wherein the rifamycins is rifampin and/or salts thereof and the tetracycline is minocycline and/or salts thereof.

32. The article of claim 15, wherein the polymeric base material is selected from the group consisting of polylactic acid, polylactides, interpolymers and copolymers thereof.

33. The article of claim 15, wherein the polymeric base material is poly(D,L-lactide-co-glycolide).

34. The article of claim 33, wherein the poly(D,L-lactide-co-glycolide) has a mole percentage of D,L lactide of about 1% to about 100%.

35. The article of claim 33, wherein the poly(D,L-lactide-co-glycolide) has a mole percentage of D,L lactide of about 40% to about 80%.

36. The article of claim 33, wherein the poly(D,L-lactide-co-glycolide) has a molecular weight of about 1 to about 200 kDa.

37. The article of claim 33, wherein the poly(D,L-lactide-co-glycolide) has a molecular weight of about 75 kDa to about 125 kDa.

38. The article of claim 15, wherein the one or more antimicrobial agents are dispersed within the polymeric base material at a concentration of about 0.01% by weight to about 50% by weight to weight of the polymeric base material.

39. The article of claim 15, wherein the one or more antimicrobial agents are dispersed within the polymeric base material at a concentration of about 15% by weight to about 25% by weight to weight of the polymeric base material.

40. The article of claim 1, wherein the antimicrobial composition is used for systemic antimicrobial therapy.

41. The article of claim 2, wherein the biological tissue is selected from the group consisting of allograft tissue, autograft tissue and xenograft tissue.

42. The article of claim 2, wherein the biological tissue is selected from the group consisting of cortical bone, cancellous bone, demineralized bone, connective tissue, tendon, pericardium, dermis, accellular dermis, cornea, dura matter, fascia, heart valve, ligament, capsular graft, cartilage, collagen, nerves, placental tissue, and combinations thereof.

43. The article of claim 3, wherein the medical device is placed temporarily.

44. The article of claim 3, wherein the medical device is placed permanently.

45. The article of claim 1, wherein the D-alpha hydroxy acid is selected from the group consisting of D-alpha hydroxy acid, its esters, its salts, its amides, derivatives thereof, and combinations thereof.

46. A method of inhibiting microbial growth on one or more surfaces of a physical object by applying to the surface an antimicrobial composition comprising a synergistic combination of a D-alpha hydroxy acid and one or more antimicrobial agents.

47. The method of claim 46, wherein the physical object is selected from the group consisting of a medical device, a biological tissue, a table, an industrial surface, a household surface, a medical surface, and combinations thereof.

48. The method of claim 46, wherein the physical object is a medical device.

49. The method of claim 46, wherein the one or more antimicrobial agents is selected from the group consisting of antibiotics, antimicrobials, antiseptics, antifungals and combinations thereof.

50. The method of claim 46, wherein the one or more antimicrobial agents is selected from the group consisting of bisbiguanide, silver nanoparticles, silver nitrate, silver oxide, silver salts, silver sulfadiazine, silver zeolites, triclosan, antifolates, aminoglycosides, carbapenems, cephalosporins, fluoroquinolines, glycopeptides, macrolides, monobactams, oxazolidones, penicillin, rifamycins, sulfonamide, tetracycline, salts thereof and combinations thereof.

51. The method of claim 50, wherein the one or more antimicrobial agents is bisbiguanide and wherein the bisbiguanide is selected from the group consisting of chlorhexidine, alexidine and combinations thereof.

52. The method of claim 50, wherein the one or more antimicrobial agents is in a form of a salt.

53. The method of claim 46, wherein the medical device is selected from the group consisting of an instrument, an implant, a device, an apparatus, and a tool.

54. The method of claim 46, wherein the medical device is reusable.

55. The method of claim 46, wherein the medical device is disposable.

56. The method of claim 46, wherein the medical device comprises a material is selected from the group consisting of metal, plastic, glass, polymeric, elastomeric, and combinations thereof.

57. The method of claim 46, wherein the synergistic combination of the D-alpha hydroxy acid and the one or more antimicrobial agents is covalently bound to the one or more surfaces of the physical object.

58. The method of claim 46, wherein the synergistic combination of the D-alpha hydroxy acid and the one or more antimicrobial agents is ionically bound to the one or more surfaces of the physical object.

59. The method of claim 46, wherein the synergistic combination the D-alpha hydroxy acid and the one or more antimicrobial agents is passively adsorbed to the one or more surfaces of the physical object.

60. The method of claim 46, wherein the D-alpha hydroxy acid is within a polymer capable of releasing D-alpha hydroxy acid monomers.

61. The method of claim 60, wherein the polymer is selected from the group consisting of polycaprolactones, polyethylene glycols, polyhydroxyalkanoates, polyesteramides, polyglycolides, polyorthoesters, polyoxazolines, polyurethanes and combinations thereof.

62. The method of claim 60, wherein the one or more antimicrobial agents is dispersed in the polymer.

63. The method of claim 46, wherein the D-alpha hydroxy acid is in the form of a polymeric base material.

64. The method of claim 63, wherein the polymeric base material comprises a polymer of the D-alpha hydroxy acid derived monomers.

65. The method of claim 64, wherein the one or more antimicrobial agents is dispersed in the polymeric base material.

66. The method of claim 63, wherein the one or more antimicrobial agent is primarily released from the polymeric base material by biodegradation of the polymeric base material and not by diffusion.

67. The method of claim 66, wherein the biodegradation of the D-alpha hydroxy acid and the release of the one or more antimicrobial agents occur at similar rates.

68. The method of claim 46, wherein the D-alpha hydroxy acid is in monomeric form.

69. The method of claim 46, wherein the D-alpha hydroxy acid is D-lactic acid and/or salts thereof.

70. The method of claim 46, wherein the one or more antimicrobial agents is a bisbiguanide.

71. The method of claim 70, wherein the bisbiguanide is in monomeric form.

72. The method of claim 70, wherein the bisbiguanide is in polymeric form.

73. The method of claim 70, wherein the bisbiguanide is chlorhexidine and/or salts thereof.

74. The method of claim 46, wherein the one or more antimicrobial agent comprises an agent selected from the group consisting of rifamycins and an agent selected from the group consisting of tetracyclines.

75. The article of claim 74, wherein the rifamycins is rifampin and/or salts thereof and the tetracycline is minocycline and/or salts thereof.

76. The method of claim 63, wherein the polymeric base material is selected from the group consisting of polylactic acid, polylactides, interpolymers and copolymers thereof.

77. The method of claim 63, wherein the polymeric base material is poly(D,L-lactide-co-glycolide).

78. The method of claim 77, wherein the poly(D,L-lactide-co-glycolide) has a mole percentage of D,L lactide of about 1% to about 100%.

79. The method of claim 77, wherein the poly(D,L-lactide-co-glycolide) has a mole percentage of D,L lactide of about 40% to about 80%.

80. The method of claim 77, wherein the poly(D,L-lactide-co-glycolide) has a molecular weight of about 1 to about 200 kDa.

81. The method of claim 77, wherein the poly(D,L-lactide-co-glycolide) has a molecular weight of about 75 kDa to about 125 kDa.

82. The method of claim 63, wherein the one or more antimicrobial agents are dispersed within the polymeric base material at a concentration of about 0.01% by weight to about 50% by weight to weight of the polymeric base material.

83. The method of claim 63, wherein the one or more antimicrobial agents are dispersed within the polymeric base material at a concentration of about 15% by weight to about 25% by weight to weight of the polymeric base material.

84. The method of claim 47, wherein the biologic tissue is selected from the group consisting of allograft tissue, autograft tissue and xenograft tissue.

85. The method of claim 47, wherein the biologic tissue is selected from the group consisting of cortical bone, cancellous bone, demineralized bone, connective tissue, tendon, pericardium, dermis, accellular dermis, cornea, dura matter, fascia, heart valve, ligament, capsular graft, cartilage, collagen, nerves, placental tissue, and combinations thereof.

86. The method of claim 47, wherein the medical device is placed temporarily.

87. The method of claim 47, wherein the medical device is placed permanently.

88. The method of claim 46, wherein the D-alpha hydroxy acid is selected from the group consisting of D-alpha hydroxy acid, its esters, its salts, its amides, derivatives thereof, and combinations thereof.

89. A method to treat or prevent disease in an animal comprising administering an antimicrobial composition comprising a synergistic combination of a D-alpha hydroxy acid and an antimicrobial agent to the animal.

90. The method of claim 75, wherein the antimicrobial composition is administered systemically.

91. The method of claim 75, wherein the antimicrobial composition is administered systemically prophylactically.

92. The method of claim 75, wherein the antimicrobial composition is administered locally.

93. The method of claim 75, wherein the antimicrobial composition is administered locally prophylactically.

94. The method of claim 75, wherein the antimicrobial composition is administered topically.

95. The method of claim 75, wherein the antimicrobial composition is administered topically prophylactically.

96. The method of claim 75, wherein the one or more antimicrobial agents is selected from the group consisting of bisbiguanide, silver nanoparticles, silver nitrate, silver oxide, silver salts, silver sulfadiazine, silver zeolites, triclosan, antifolates, aminoglycosides, carbapenems, cephalosporins, fluoroquinolines, glycopeptides, macrolides, monobactams, oxazolidones, penicillin, rifamycins, sulfonamide, tetracycline, salts thereof, and combinations thereof.

97. The method of claim 75, wherein the animal is selected from the group consisting of a food production animal, a companion animal, a working animal and a human.

98. The method of claim 83, wherein the animal is a human.

99. The method of claim 89, wherein the D-alpha hydroxy acid is within a polymer capable of releasing D-alpha hydroxy acid monomers.

100. The method of claim 99, wherein the polymer is selected from the group consisting of polycaprolactones, polyethylene glycols, polyhydroxyalkanoates, polyesteramides, polyglycolides, polyorthoesters, polyoxazolines, polyurethanes and combinations thereof.

101. The method of claim 99, wherein the one or more antimicrobial agents is dispersed in the polymer.

102. The method of claim 89, wherein the D-alpha hydroxy acid is in the form of a polymeric base material.

103. The method of claim 102, wherein the polymeric base material comprises a polymer of the D-alpha hydroxy acid derived monomers.

104. The method of claim 102, wherein the one or more antimicrobial agents is dispersed in the polymeric base material.

105. The method of claim 102, wherein the one or more antimicrobial agent is primarily released from the polymeric base material by biodegradation of the polymeric base material and not by diffusion.

106. The method of claim 102, wherein the biodegradation of the D-alpha hydroxy acid and the release of the one or more antimicrobial agents occur at similar rates.

107. The method of claim 89, wherein the D-alpha hydroxy acid is in monomeric form.

108. A method of inhibiting microbial growth on one or more surfaces comprising applying an antimicrobial combination comprising a synergistic combination of a D-alpha hydroxy acid and an antimicrobial agent to the one or more surfaces.

109. The method of claim 108, wherein the one or more surfaces is selected from the group consisting of an industrial surface, a household surface, and a medical surface.

110. An antimicrobial composition comprising a synergistic combination of a D-hydroxy acid and one or more antimicrobial agents.

111. The antimicrobial composition of claim 110, wherein the D-alpha hydroxy acid is within a polymer capable of releasing D-alpha hydroxy acid monomers.

112. The antimicrobial composition of claim 111, wherein the polymer is selected from the group consisting of polycaprolactones, polyethylene glycols, polyhydroxyalkanoates, polyesteramides, polyglycolides, polyorthoesters, polyoxazolines, polyurethanes and combinations thereof.

113. The antimicrobial composition of claim 111, wherein the one or more antimicrobial agents is dispersed in the polymer.

114. The antimicrobial composition of claim 110, wherein the D-alpha hydroxy acid is in the form of a polymeric base material.

115. The antimicrobial composition of claim 114, wherein the polymeric base material comprises a polymer of the D-alpha hydroxy acid derived monomers.

116. The antimicrobial composition of claim 114, wherein the one or more antimicrobial agents is dispersed in the polymeric base material.

117. The antimicrobial composition of claim 114, wherein the one or more antimicrobial agent is primarily released from the polymeric base material by biodegradation of the polymeric base material and not by diffusion.

118. The antimicrobial composition of claim 117, wherein the biodegradation of the D-alpha hydroxy acid and the release of the one or more antimicrobial agents occur at similar rates.

119. The method of claim 110, wherein the D-alpha hydroxy acid is in monomeric form.
